(19) United States
(12) Patent Application Publication
(10) Pub. No.: US 2019/0059209 A1
(43) Pub. Date: Feb. 28, 2019

(54) HIGH RESOLUTION SOIL ROOTING ZONE PENETROMETER

(71) Applicant: PIONEER HI-BRED INTERNATIONAL, INC., Johnston, IA (US)

(72) Inventors: Philip F. Brune, Parkland, FL (US); Travis Kriegshauser, Urbandale, IA (US)

(73) Assignee: PIONEER HI-BRED INTERNATIONAL, INC., JOHNSTON, IA (US)

(21) Appl. No.: 16/102,415

(22) Filed: Aug. 13, 2018

Related U.S. Application Data
(60) Provisional application No. 62/587,787, filed on Nov. 17, 2017.
(60) Provisional application No. 62/563,163, filed on Sep. 26, 2017.
(60) Provisional application No. 62/549,827, filed on Aug. 24, 2017.

(51) Int. Cl. A01C 7/20; A01B 63/111; G01N 33/24

(52) U.S. Cl. CPC A01C 7/203; G01N 2033/245; G01N 33/24; A01B 63/1115

(57) ABSTRACT

An apparatus and methods for analyzing the soil rooting zone for agricultural crops in high resolution to determine the mechanical resistance and related physical, mechanical and hydrological properties, and uses of this information in crop production. Uses include crop selection, real time seeding rate determination, field management prescriptions, yield prediction, assessment of root lodging risk, and real time planting depth determination.

### TECHNICAL FIELD

Embodiments of the present disclosure relate to an apparatus and methods for analyzing the soil rooting zone for agricultural crops in high resolution to determine information regarding soil physical, hydrological, and mechanical properties, and uses of this information in crop production.

### BACKGROUND

There is a need to quickly and accurately assess soil physical, hydrological, and mechanical properties within the soil rooting zone of agricultural crops and to use this information for crop production.

### SUMMARY

An apparatus and methods for analyzing the soil rooting zone for agricultural crops in high resolution to determine the mechanical resistance and related physical, mechanical and hydrological properties, and uses of this information in crop production. Uses include crop selection, real time seeding rate determination, field management prescriptions, yield prediction and assessment of root lodging risk.

### BRIEF DESCRIPTION OF THE DRAWINGS

The present disclosure is illustrated by way of example, and not by way of limitation, in the figures of the accompanying drawings and in which:

FIG. 1 graphically shows farm equipment, in this embodiment a planter, with the penetrometer mounted on the farm equipment in the direction of travel ahead of the planting assembly.

FIG. 2 shows is a close-up side view of the penetrometer, which in this embodiment, is located on the row cleaner mount of a planter.

FIG. 3 shows a top view of the penetrometer shown in FIG. 2.

FIG. 4 shows a front view of the penetrometer shown in FIG. 2.

FIG. 5 shows a side view of an alternative embodiment of the penetrometer, with the addition of axial springs to allow for depth changes, as well as axial load cells (21) to measure the mechanical resistance of the soil interacting with the penetration wheel.

FIG. 6 shows a close-up side view of one penetration cone operably connected to a load cell mounted on the penetration wheel.

FIG. 7 shows a side view of the bottom of the extension bar at the location where the penetration wheel is connected, and, in this embodiment, an accelerometer positioned at a rotational point of the penetration wheel

FIG. 8 shows a close-up side view of the axial springs and axial load cells, also shown in FIG. 5, that are positioned within the extension bar.

FIG. 9 shows a flow chart representing one embodiment in which the penetrometer is integrated with a planting rate control system, which is used to alter seed planting density in response to the soil measurement obtained by the penetrometer.

FIG. 10 is a schematic illustration of the quantities used to define the anchorage sub-model. The assumed failure interface is the outer extent of the root-reinforced soil region, shown at radius (R).

FIG. 11 is a schematic illustration of the field stalk angle (θ) following a root lodging event, measured at the base of the plant and used to quantify the severity of lodging.

FIG. 12 shows the penetrometer of FIG. 1, modified to show an opening disc and closing disc.

### DEFINITIONS

“Row Cleaning mount” means a part that attaches a row cleaner to a planter row unit.

“Conical” means, broadly, any shape generally approaching a conical form, and includes frustoconical shapes, pyramids, and 5, 6, 7, 8, 9, 10 sided objects narrowing in three dimensional space to a point or blunt end capable of being inserted into the soil of any agricultural field.

“Data file” means an electronic file that contains numerical data.

“Grain” means the harvested seeds of a row crop used for food.

“Farm vehicle” means any machinery capable of traversing a field, including but not limited to a planter, tractor, rover, harvester, all-terrain vehicle, sprayer, or fertilizer.

“Finite Element Node” means a vertex of a finite element, where structural displacements are calculated by finite element analysis, which involves dividing a domain into smaller simpler subdomains.

“Planting density” means the as-planted plant population rate of a crop, typically measured in plants/acre.

“Protruding Surface” means a part that extends outward from a larger part.

“Proximal to” means nearby spatially within a relevant length scale.

“Real time planting adjustment” means a change in the seed planting depth, the variety planted and/or the seed planting density as the planter is in the field, and preferably, as the planter is moving through the field.

“Root lodging” means the irreversible mechanical deformation of a plant's subterranean support structure. It is a physical process by which wind action on a plant's above-ground structure generates an aerodynamic load, whose resultant bending moment surpasses the root-soil anchorage capacity, causing a rotation of the below-ground support base and angling the plant stalk from a vertical position.

“Saturated hydraulic conductivity” means a quantitative measure of a saturated soil's ability to transmit water when subjected to a hydraulic gradient. It is a measure of the ease with which pores of a saturated soil permit water movement.

“Seed Planting Mechanism” means a device that opens a furrow in field soil and deposits a seed therein.

“Soil bulk density” means the mass per unit volume of dry soil.

“Soil cohesion” means the component of soil shear strength that arises from electrostatic bonds between smaller particles (e.g. silt and clay) and/or capillary forces in water menisci that bridge particles.

“Soil compaction” means the densification of soil due to displacement of air from pores between soil grains.

“Soil consolidation” means the densification of soil due to displacement of water from pores between soil grains.

“Soil hydrological properties” mean the saturated hydraulic conductivity and/or surface-level water holding capacity of soil.

“Soil interacting part” means, with respect to the penetrometer described herein, a part of the penetrometer that is in direct contact with the soil, such as the penetration cone. A direct connection between a load cell and a soil interacting part occurs when there is no intermediate linkage that distributes, transfers, or alters the load between the soil interacting part and the load cell.

“Soil mechanical resistance” means the ability of soil to retain its structure when subjected to mechanical forces arising from interaction with external bodies. The mechanical resistance is a composite measure that implicates several other soil properties, including soil bulk density, soil (volumetric) moisture, soil shear strength, soil cohesion, and susceptibility to soil compaction and soil consolidation.

“Soil moisture content” means the extent to which pores between soil particles are filled with water, and can be defined volumetrically or gravimetrically.

“Soil properties” means soil properties such as soil mechanical resistance, soil hydrological properties, soil moisture content and the like.

“Soil shear strength” means the magnitude of shear stress that a soil can sustain, arising from interparticle friction, interlocking, and soil cohesion.

### DETAILED DESCRIPTION

In vascular plants, the root is the organ of a plant that typically lies below the surface of the soil. It is a non-leaf, non-node bearing part of the plant's body that is important for many aspects of plant growth. However, the root architecture, or spatial configuration of the plant's root system, plays an important structural function as well, as it physically anchors the plant to the soil. When strong winds or other lateral forces cause a plant to tip over or fall entirely to the ground, this causes complete or partial yield loss. Even if mature grain is present on the plant, modern harvesting equipment may not be able to properly harvest the grain.

In certain varieties of annual crops grown for commercial grain production, root zones tend to be concentrated in the top four inches of the soil, where nutrient availability and aeration are more favorable for growth. In crops such as corn, brace roots develop to help anchor the plant. Root architecture varies among different varieties, and root lodging resistance has commonly been used by plant breeders as a trait that is selected for during the breeding process. Growers will then select a variety on the basis of its lodging resistance if they view this as a perceived need for a particular field.

Fields and management zones are commonly viewed based on soil types, such as various combinations of sand, silt and clay. For example, the USDA SSURGO database contains information about soil types collected by the National Cooperative Soil Survey over the course of a century. In many cases, the information was obtained by laboratory analysis of soil samples. Each map unit of information may contain one to three major components and some minor components. The map units are typically named for the major components. Examples of information available from the database include available water capacity, soil reaction, electrical conductivity, and frequency of flooding; yields for cropland, woodland, rangeland, and pastureland; and limitations affecting recreational development, building site development, and other engineering uses.

In contrast, embodiments of the invention view root lodging from a different, more structural engineering perspective. The embodiments were developed based on viewing an individual plant's resistance to lodging as relating to structural failure of the root-soil anchorage system that adversely affects the plant's yield. Soil mechanical resistance, and in particular soil mechanical strength, was evaluated as a major determinant of root lodging, without direct consideration of soil type. Soil mechanical resistance was evaluated at a much higher resolution than soil type differences, which allows use of this information to determine crop selection, real time seeding rate determination, field management prescriptions, and assessment of lodging risk. Further, a device to determine soil mechanical resistance could be placed on the same farming equipment conducting a farming operation, thereby enabling real time farm management decisions. For example, by placing a penetrometer capable of taking high frequency real time measurements ahead of a planter, real time planting density can be changed to account for the actual soil mechanical resistance in the location in which the seed is being planted.

Root lodging is a complex phenomenon that depends strongly on both crop genetics and environmental factors. An accurate biomechanical model was developed that takes into account the soil physical, mechanical, and soil hydrological properties, the stability of the root architecture of that plant in the soil, and wind force, neighboring plants, and other directional stabilizing and destabilizing forces acting upon the plant. From this perspective, a given plant variety's genetic characteristics are viewed as fixed, while the soil characteristics, measured by a rating from hard to soft, are viewed as a changing variable. This change of perspective, to a focus on the measurement of the soil to determine the soil mechanical resistance, enables subsequent decisions, such as variety selection, planting density, and even crop type, to be based upon the measured soil mechanical resistance, even in cases where the soil type is not known. In cases where soil type is known, hyperlocal differences in the actual measured soil mechanical resistance of the soil can be detected and used to make more informed real-time or later in time farm management decision.

While soil probes to measure soil mechanical resistance are known, they typically consist of a single probe that is inserted into the soil. The probe taking instrument must be stopped and positioned as the probe is inserted, often taking a measurement at several points of depth, including measurements well below the top four inches of the soil, which is the primary structural rooting zone for many crops. This process typically results in a fairly low resolution view of soil mechanical resistance. Thus, measurements may be sparse and non-continuous, and are time consuming to take. Embodiments of the present invention include a penetrometer designed specifically to take fast and continuous measurement of the soil mechanical resistance in the primary crop rooting zone. The penetrometer may be mounted on any suitable farm vehicle traveling through the field, including but not limited to a tractor, rover, harvester, all-terrain vehicle, sprayer, or fertilizer. In one embodiment, the penetrometer is mounted on a planter. When mounted on a planter, the penetrometer may provide real time information on soil conditions that are used to determine planting density on the go. In one embodiment illustrated herein, the penetrometer is mounted on the leading edge of the planter, thereby providing a measurement of the soil conditions ahead of the planting device. This provides an advantage over a conventional penetrometer, load sensing pin or probe utilized by planter seed planting mechanisms to determine planting depth, because this permits planting density to be optimized based on the real time measurement of the soil conditions in the field location where the seeds will be planted. Multi-variety planters may be used in conjunction with this embodiment as well, thereby allowing the grower or a computer to adjust the variety and/or planting density based on the real time measurement of the field soil conditions.

### Penetrometer

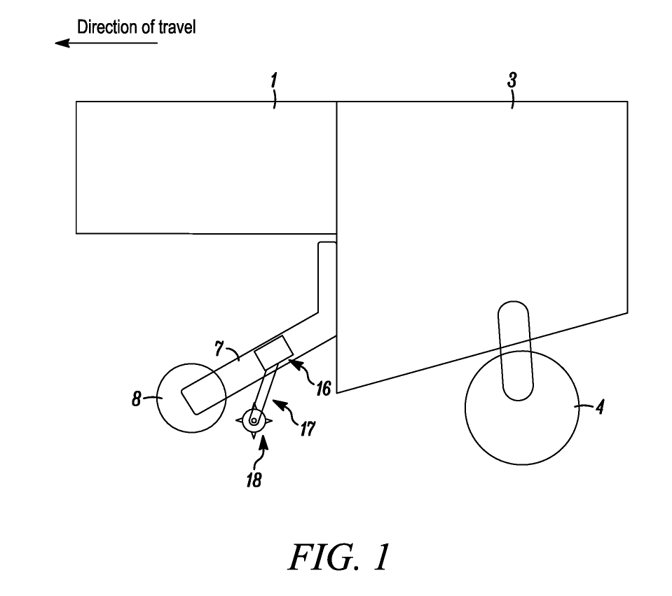

In one embodiment, as shown in FIG. 1, the penetrometer is positioned on the planter's row cleaner mount (7), in the direction of travel ahead of the planting assembly (4). This mount (7) holds a row cleaner implement (8) that clears debris from the row ahead of planting, and provides a convenient location in which to place the penetrometer for measurement of the soil properties. Also shown (symbolically) in FIG. 1 are the planter frame (1) and the row unit frame (3). In this embodiment, the penetrometer is shown comprising a drive motor (16), extension bar (17) which connects the drive motor belt or chain (not shown) so that it may turn the penetration wheel (18). The penetrometer may be positioned at any distance ahead of the planter gauge wheel, including but not limited to at least a quarter meter, a half meter, one meter, two meters, three meters, four meters, five meters, etc. ahead of the gauge wheel. In certain embodiments, the mounting distance should provide sufficient time to make real time planting density calculations and corresponding planting assembly adjustments.

In another embodiment, the measurements from the penetrometer may be fed into a crop model, which may be run to determine a variety or varieties that would be suitable for a given crop type. This model may be run in real time to determine a variety to plant when using a multi-variety planter. Alternatively, this data could be collected at harvest or at other times and used to select the variety to be planted in a particular field location.

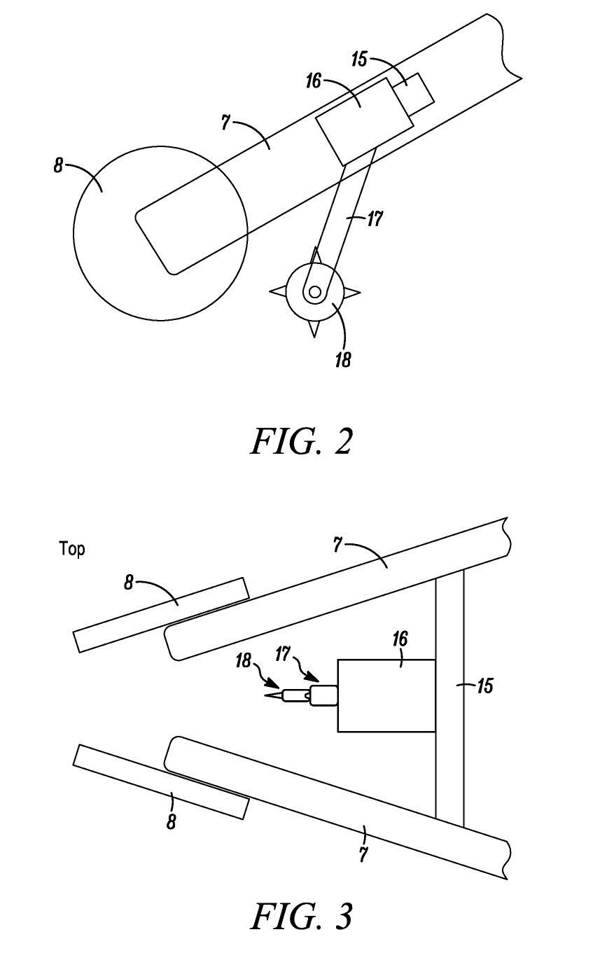

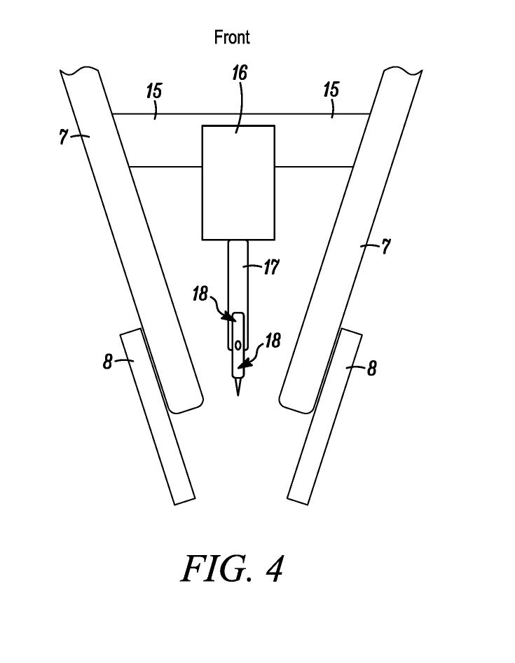

Referring to the embodiment shown in FIG. 2, the penetrometer is comprised of a drive motor (16), an extension bar (17), and a penetration wheel (18). As can be seen in this figure, and in additional detail in FIG. 3 and FIG. 4, the drive motor (16), an extension bar (17), and a penetration wheel (18) may be mounted on a mounting crossbar (15). The row cleaner mount (7) and row cleaner implement (8) may extend in front of the penetrometer, thereby clearing the field to provide a cleaner field surface for measurement (and planting). The row cleaner mount (7) and row cleaner implement (8) may be optimized to any length or angle to best fit the geometry of the farm equipment relative to the soil.

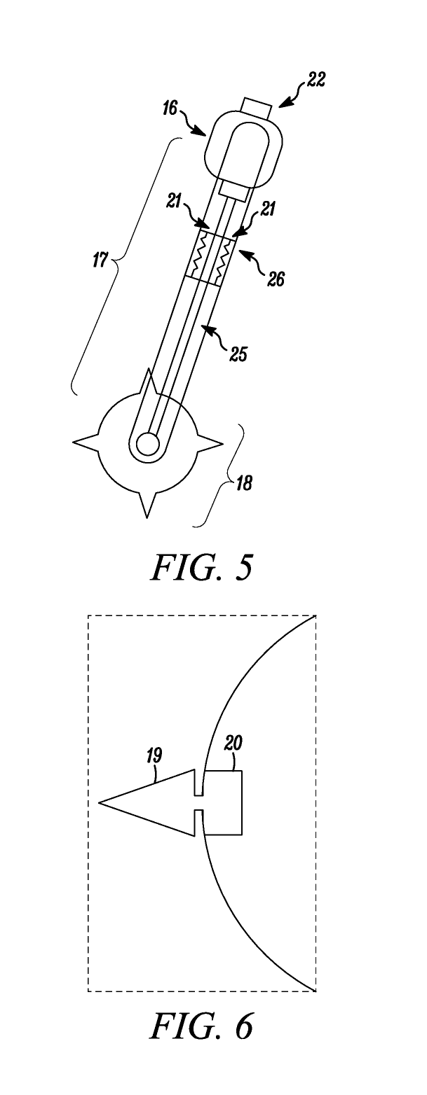

FIG. 5 shows a more detailed view of an alternative embodiment of the penetrometer, with one or more load cells, the axial load cells (21) positioned above the spring or springs to provide measurements of the force upon the penetration wheel (18) as it moves through the soil. In such embodiment, an electronic drive motor (16) powers a drive shaft (25) that is mounted within an extension bar (17). An electronic power meter (22) is connected to the drive motor (16), which can be used to measure the of electric power consumed by the drive motor (16), which is controlled to rotate at a constant number of revolutions per minute, subject to changes in planter velocity and soil mechanical resistance, in order to ensure penetration data at regular spatial intervals. This measure of power may also serve as an indirect measure of the force required to insert and turn the penetration wheel (18) as it moves through the soil. The drive shaft (25) is operably connected to the penetration wheel (18), and may be connected by any suitable means, including gears, belts or chains.

As seen in FIG. 6, the penetration wheel (18) itself also comprises a series of load cells (20) mounted behind a penetration cone (19), which cone as embodied here is conical, but may be any number of shapes and sizes. In the embodiments shown in FIG. 5, four penetration cones (19) are shown on the penetration wheel (18). However, the number of circumferentially installed penetration cones can vary, such as from 1 to 100, or any whole number in between. The shape of the cones may be altered to teeth, tines, or any other shape that may be quickly inserted and removed from the soil. Further, in alternate embodiments, penetration wheel (18) may be a rolling mechanism with a different configuration, such as a rolling drum, a coulter disk, or a flanged disk. Any number and type of commercially available load cells, load sensing pin, or similar may be used, including but not limited to hydraulic load cells, pneumatic load cells and strain gauge load cells. In this embodiment, a miniature button load cell is shown.

Once the penetration cone (19) or similar shape contacts the soil, the penetration wheel load cell (20) in contact with the soil will register the force generated by the penetration cone (19) as the penetration cone (19) directly presses on or into the soil. This provides a more direct measurement of soil mechanical hardness than load cells or load sensing pins that are connected via a linkage, such as the load sensing pin described in US 2012/0180695. The penetration wheel load cells will result in frequent measurements of the soil roughly commensurate in depth with the conical shape used. For example, if four two inch cones were equally spaced on a wheel with a 16-inch circumference, then a measure of the soil depth 2 inches below the perimeter of the penetration wheel (18) would be obtained every 4 inches. This would be sufficient to create a very high resolution map of the soil mechanical resistance, and is roughly commensurate with seed spacing of about 5 inches, which is the approximate spacing of seeds at a planting density of 32,000 seeds per acre with 30 inch rows. The circumference of the penetration wheel (18) and the number and dimensions of the penetration cones (19) can be easily varied by one of ordinary skill in the art to suit alternative planting densities and/or crops.

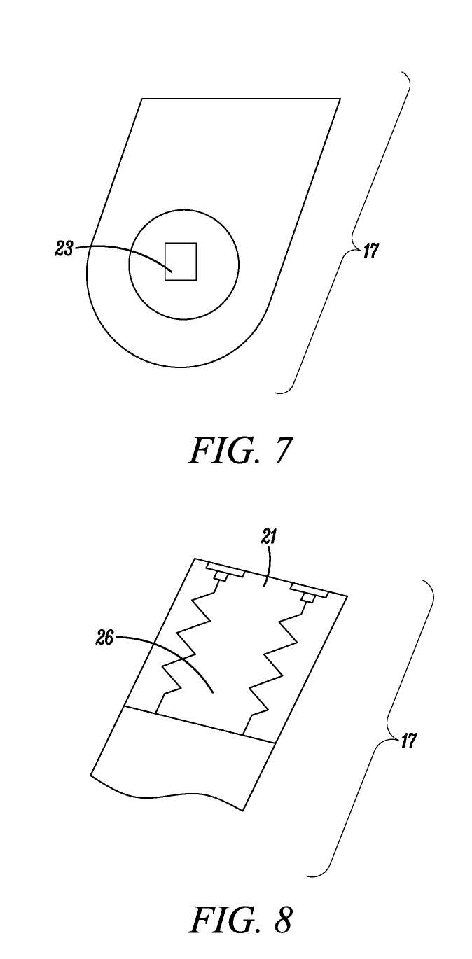

In an optional embodiment, described briefly above and shown in more detail in FIG. 8, axial springs (26) are positioned within the extension bar to allow for the arm to change in depth to keep the penetration wheel (18) in contact with the ground during operation. Axial load cells (21) are positioned above the springs to measure the force transmitted through the extension bar (17). This axial force can either provide additional information about local variations in depth acting upon the wheel and arm that can be applied to improve the data obtained from the load cells, or provide information about the soil mechanical resistance on a larger length scale than that of the relatively smaller penetration cone (19) and load cell (20). Relatedly, as seen in FIG. 7, an accelerometer (23) may be positioned at a rotational point at the base of penetration wheel (18) to provide additional information about changes in depth caused by the interaction of the penetration wheel with the soil.

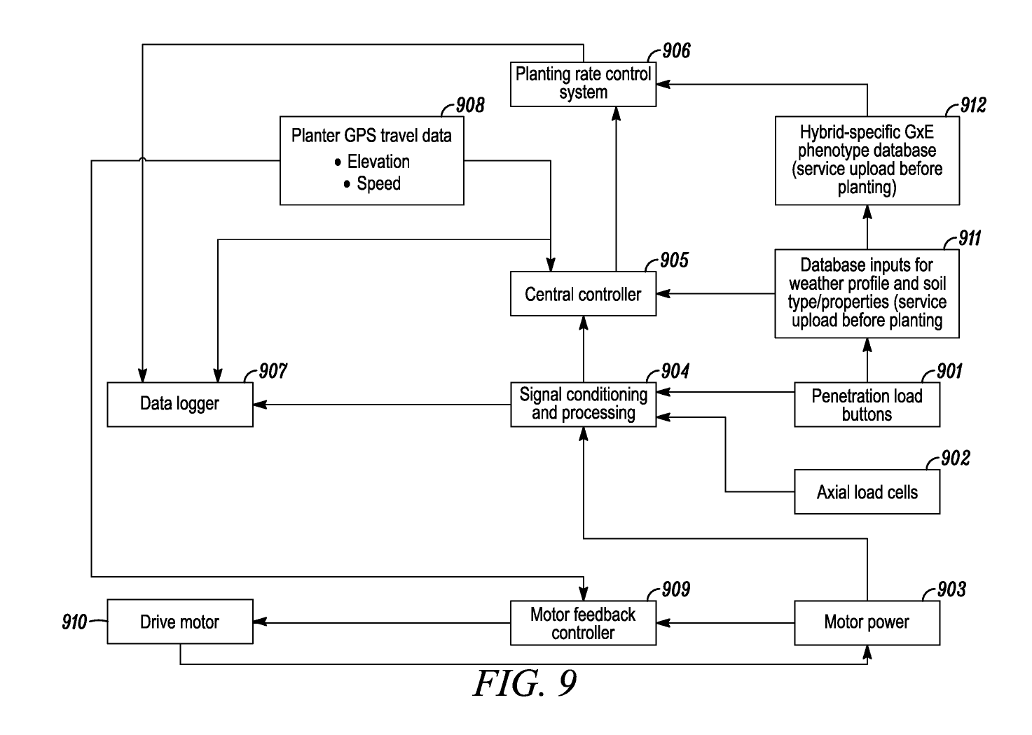

As shown in FIG. 9, the penetrometer may be used to determine an optimum planting rate in real time as planting occurs. Electronic signals from the penetrometer load cells (901)(902), as well as a measure of the power of the motor (903) used to drive the rolling mechanism may be transmitted to a signal conditioning and processing unit (904), which simultaneously outputs the processed data to a logger (907) and, optionally, enriches it with hybrid and location specific data, such as inputs for weather profiles and soil type/properties (911), to arrive at a quantitative optimum planting density. The hybrid and location specific data (912) may be uploaded from a service database before planting in order to provide real time planting density determinations while avoiding any data upload or download delays. The optimum planting density is passed as an instruction set to the planting rate control system (906), which can then dynamically vary the planting rate to achieve the calculated optimum density. The central controller (905) is used to gather the conditioned and processed signals (904) from the load cells (901) (902) and motor (903), as well as the weather and soil type/properties (911) and Planter GPS travel data, such as elevation and speed (908). The drive motor (910) may utilize a motor feedback controller (909) to vary the power to the motor (903).

Additional embodiments of the penetrometer (not shown) may also include a component to measure local changes in elevation and/or depth of the penetration wheel vial an onboard ultrasonic or laser sensor system. A soil moisture probe may be added to the system to provide real time data on soil moisture conditions. Other probes may be added to provide information on soil hydrological conditions.

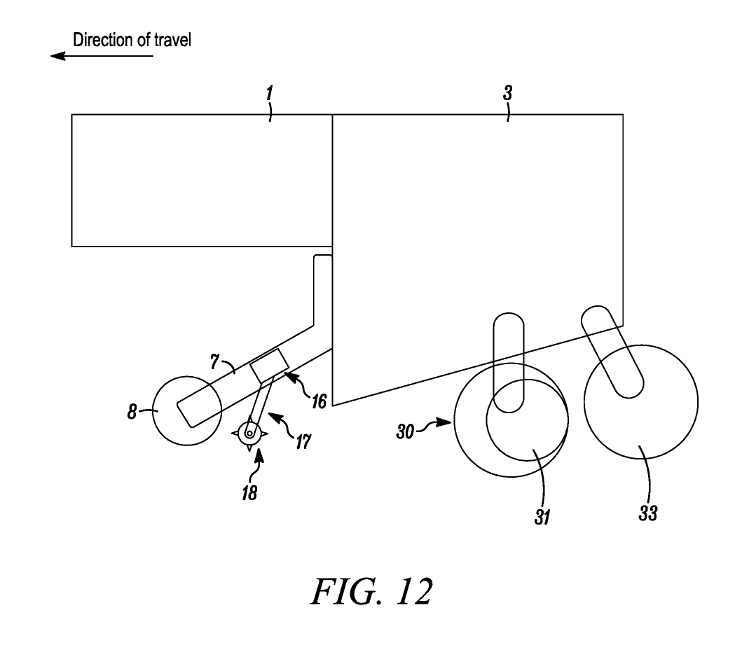

In conjunction with a soil moisture probe and/or depth measurement, the penetrometer described herein can be utilized to determine the optimal real time planting depth for the soil conditions as the planter moves through the field and identifies different soil and moisture conditions. This is ideal for developing a uniform stand throughout the field, in order to establish similar periods of germination, silking, pollination, nick, seed development and dry down. Planting may be optimized to plant the seed at a depth, typically at the top of the moisture layer, that ensures good seed-soil contact and a moisture level sufficient to enable to seed to imbibe about 30% of its weight in water to germinate. Planting depth and/or furrow width may be optimized based on one or both of soil mechanical resistance, as determined by the penetrometer, and soil moisture, as determined by the soil moisture sensor. The methods and systems of using the penetrometer to establish planting depth in real time may be further synchronized to work in conjunction with the planter's opening discs and/or closing discs. See FIG. 12 for a graphical illustration of a planting assembly comprising an opening disc (30) and gauge wheel (31), and closing discs (33). Adjustable closing discs are well known, for example, see U.S. Pat. No. 4,570,554. Such adjustable closing discs may be automated by one of ordinary skill in the art, such as by using hydraulic down pressure, pneumatic down pressure and/or electromechanical springs to adjust closing disc depth and width. In a fully automated system, the penetrometer and/or moisture sensor would determine an optimal planting depth and/or furrow width, to which the opening discs, planter mechanism and closing discs would automatically respond.

### Biomechanical Model

The biomechanical model developed for use in conjunction with the penetrometer described above, or which can be used with any other type of penetrometer, load sensing pin, or similar, employs an engineering safety factor approach to quantify root lodging resistance as the ratio of anchorage supply and wind demand. Field experiments were conducted to parametrize the model for a sensitivity analysis and validate the model for predictive accuracy. Once the model is applied to the penetrometer or other soil parameter testing device, the penetrometer or other device may be calibrated to provide a direct assessment of soil mechanical resistance that will equate to a lodging risk factor. This lodging risk factor may be used immediately, in real time, by crop planting equipment to determine proper seed planting density and, optionally, seed planting depth.

Root lodging afflicts a variety of cereal crops. Broader scientific efforts have focused on wheat (Triticum aestivum L.), barley (Hordeum vulgare L.), and oats (Avena sativa L.) (Pinthus 1974; Berry et al. 2004). The present model focuses on maize (Zea mays L.), for which there is less preceding scholarship on root lodging of fully intact plants. With some exceptions (Carter and Hudelson 1988; Stamp and Kiel 1992), much of the work involves roots that have been variously compromised by corn root worm (e.g. Spike and Tollefson 1991).

Accurate modeling of maize root lodging events requires careful representation of the relevant physical phenomenology. Dynamic amplification is an essential component in the mechanical excitation of (plant) structures by wind. A steady wind at constant velocity blowing on a stably supported object applies a ‘static’ aerodynamic force through drag. If occasional gusts are superimposed atop the steady wind, then additional dynamic forces can significantly increase the load on the structure if the interval of the gusts excites a resonant frequency of the structure. In this case, the periodic dynamic loads cause large oscillatory displacements of the structure that significantly amplify the mechanical load on the structure and its supports.

Accordingly, the plant's non-dimensional root lodging resistance (RLR) may be defined as the ratio of the computed anchorage supply (AS) and wind demand (WD):

RLR≡AS/WD (1)

Both AS and WD were directly computed by sub-models as equivalent bending moments [N*mm].

### Wind Demand Sub-Model

The wind demand sub-model adopted a spectral representation of the airflow and its resulting aerodynamic loads. The model was implemented in a commercial finite element analysis platform, which facilitated more sophisticated treatments of the additional complexity presented by the maize plant structure and material, specifically taper in the elliptical stalk cross-section, variously located and sized leaves, and the difference in mechanical response of internode versus node stalk tissue.

Model creation started with generating the structural geometry. The stalk was the primary structure of interest, and was represented directly in the model. Key input parameters were total plant height [cm] and the locations of nodes along the stalk [% of height], defining the structural geometry of the stalk in terms of internode lengths and node positions. Each node was assigned a thickness value of 6.4 mm, although other values, such as those within the range of 3 mm to 9 mm, can be used to accommodate structural differences in maize germplasm. The stalk was discretized using structural beam elements with shear flexibility to represent the low aspect ratio (length/diameter) of stalk node (as opposed to internode) regions. The elliptical stalk cross-section and its taper with height were implemented via general beam sections. Both the node and internode material responses were defined as linear elastic. This material model was sufficient to represent the difference in node and internode material stiffness, accounted for in the model by a 3× increase to the elastic modulus [GPa] in the node sections following the measurements of stalk structural stiffness [N/m] in Robertson et al. (2014). A uniform mass density [gm/cc] was used for the entire stalk, as localized increases in the node sections were analytically determined not to significantly alter the responses of interest.

Other mechanically consequential features of the maize plant were represented indirectly as engineering features. Leaves were modeled by aerodynamic forces applied to the stalk nodes, with magnitudes scaled by a triangular approximation of their area [cm²]; more detail appears subsequently in the description of the model aerodynamics. Finite root-soil stiffness was represented by a torsional spring [N*mm/rad] connected to a fixed boundary. As noted in Baker (1995), including the compliance of the roots and soil was important for accurately predicting the natural frequency [Hz] of the plant; assuming a fixed boundary condition (infinite root soil stiffness) increased the computed natural frequency by ˜3×. For modeling of root lodging of mature plants, an ear was implemented as a lumped mass [gm] located at an input ear height [cm].

The aerodynamics representation approximated the transformation of turbulent wind energy into mechanical loads on the plant structure. The approach combined several components to produce a spectral representation of the aerodynamic force applied by the wind to the plant. The first component was the aerostatic force FAS [N], computed as:

$${F}_{AS} ( z ) = \frac{1}{2} ρ \cdot {A}_{A} ( z ) \cdot {C}_{d} \cdot {{V}_{avg} ( z )}^{2} \qquad ( 2 )$$

with z [cm] the vertical coordinate along the stalk, ρ0 the mass density of air [gm/cc], A_{A} the aerodynamic area [cm²], C_{d} the effective drag coefficient, and V_{avg} the average wind speed [m/s]. The aerostatic force was computed for each finite element node based on the lengths and diameters of the elements connected to it. In considering boundary value problems, the finite element method discretized the domain into a mesh of interconnected finite elements. The vertices that defined the coordinates of the elements are called nodes. They should not be confused with stalk nodes. If the finite element node was associated with a region in the internode of the stalk, the aerodynamic area was that of the associated stalk volume and the effective drag coefficient was set to the value for the stalk (C_{d}_{_}ₛ=1.0), taking the value for right circular cylinders in cross-flow with a Reynolds number below 5×10⁵. If the finite element node was associated with a region in the node of the stalk, an additional drag force associated with the leaf was superposed atop the stalk drag force. The effective drag coefficient for the leaf C_{d}_{_{—l}} was input using data from Wilson N R, Shaw R H (1977) A higher order closure model for canopy flow. Journal of Applied Meteorology 16: 1197-1205, and Flesch T K, Grant R H (1991) The translation of turbulent wind energy to individual corn plant motion during senescence. Boundary Layer Meteorology 55:161-176. The leaf aerodynamic area was determined as a function of the height of the leaf (i.e. the height of the stalk node to which it was attached) using the plant area density scaled to the height of the plant being considered, as described in Shaw R H, Den Hartog G, King K M, Thurtell G W (1974) Measurements of mean wind flow and three-dimensional turbulence intensity within a mature corn canopy. Agricultural Meteorology 13: 419-425. Additionally, a drag reduction factor of 0.5 was applied to reduce the leaf forces from skin drag, reflecting measurements that streamlined bodies experience reduced drag at higher Reynolds number flows. Finally, the distribution of average wind speed was determined as a function of the height of the stalk (z) via the normalized velocity profile. The input average wind speed from a weather station V_{avg}_{_}_{WS} was used to quantify the actual (as opposed to normalized) vertical distribution of average wind speed (Shaw et al. 1974, supra) via:

$${V}_{avg} ( z ) = {V}_{{avg}_{WS}} \cdot exp ( α \cdot ( \frac{z}{{h}_{WS}} - 1 ) ) \qquad ( 3 )$$

with α the exponential coefficient for a mature maize canopy and hws the height [m] of the weather station at which V_{avg}_{_}_{WS} was measured.

The second component used to obtain a spectral representation of the aerodynamic force was the aerodynamic admittance function Γ:

$${Γ ( f , z )}^{2} = \frac{1}{1 + 2.5 \cdot {( \frac{f \cdot {D}_{c}}{{V}_{avg} ( z )} )}^{2}} \qquad ( 4 )$$

with f the frequency [Hz] being analyzed, V_{avg} the average wind speed, and D_{c} [m] the canopy diameter, which is the periodic plan view area encompassed by the plant, and is estimated from the planting density PD [plants/acre]:

D_{c}=2\sqrt{1/PD} (5)

The aerodynamic admittance truncated the frequency spectrum of the in-canopy turbulent airflow by removing the higher frequencies whose action does not excite the vibrational modes of the plant that determine its structural response to wind gusts.

The final component used to define the force spectrum was the velocity spectrum of the wind. The Von Karman form was adopted. The velocity power spectrum density (PSD) Sᵥ [(m/s)²/Hz] was expressed as:

$${S}_{v} ( f , z ) = \frac{4 {σ}_{v}^{2} \cdot ( \frac{f \cdot {L}_{tb}}{{V}_{avg}} )}{f \cdot {( 1 + 70.8 \cdot {( \frac{f \cdot {L}_{tb}}{{V}_{avg} ( z )} )}^{2} )}^{5 / 6}} \qquad ( 6 )$$

with L_{tb} [m] the turbulence length scale and σᵥ [m/s] the standard deviation of the wind speed. The velocity PSD, aerodynamic admittance, and aerostatic force were combined to calculate the aerodynamic force PSD for each FEN as Sₚ [N²/Hz]:

$${S}_{p} ( f , z ) = 4 {S}_{v} ( f ) \cdot {( \frac{{F}_{as} ( z ) \cdot Γ ( f )}{{V}_{avg} ( z )} )}^{2} \qquad ( 7 )$$

The WD sub-model was run in two steps. The first step calculated the modal response of the plant. While only the lowest two vibration modes participated significantly in the dynamic response, the frequencies associated with the first four modes were calculated to be conservative. The second step applied a random response analysis that utilized the previously calculated modal response and the aerodynamic force PSD to determine the PSD of the resultant bending moment at the base of the plant B_{PSD} [(N*mm)²/Hz]. The total effective bending moment Bₘₐₓ [N*mm] at the plant base was then calculated by summing the dynamic and static contributions:

$${B}_{max} = {\sum }_{i} {{F}_{AS} ( {z}_{i} )}_{i} \cdot {z}_{i} + GF \cdot \sqrt{\int {B}_{PSD} df} \qquad ( 8 )$$

The first term was the static component, obtained by summing the bending moments generated by the aerostatic force applied at each FEN. The second term was the dynamic component, calculated as the root mean square of the bending moment PSD scaled by a gust factor; the gust factor was defined as a constant value of 4, although values ranging from 2 to 20 may be used. The maximum bending moment is the output of the WD sub-model.

### Anchorage Supply Sub-Model:

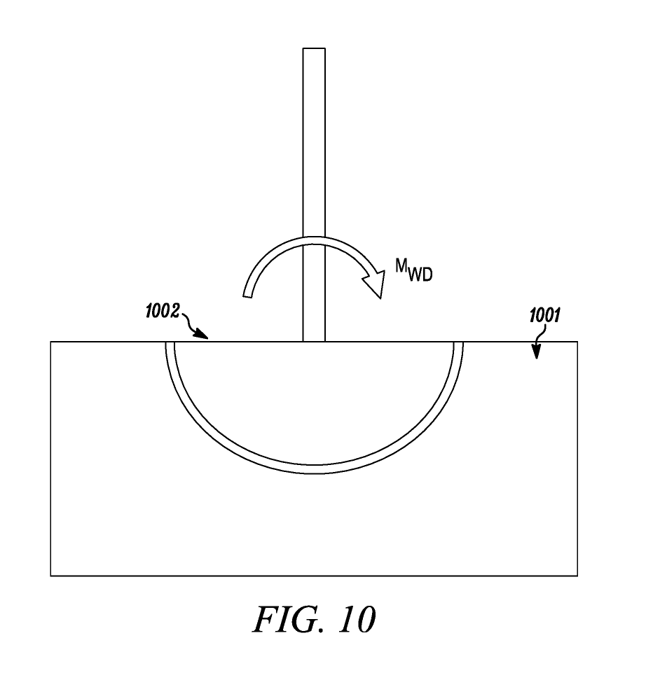

The anchorage supply sub-model followed a more straightforward mechanistic approach. It was developed from a closed-form analytical representation of the anchorage zone. The anchorage zone was modeled as a region of bulk soil (1001) surrounding a hemi-spheroid of root-reinforced soil (1002) that approximated the maize root ball and was subjected to an applied bending moment (FIG. 10). Anchorage failure was described as a rotation of the root-reinforced soil volume along the interfacial surface between bulk and root-reinforced soil. This rotation was resisted by the soil shear strength of the interface, assumed to be the total shear strength of the bulk soil, τ [kPa], expressed in Mohr-Coulomb form as:

τ=c+σ·tan ϕ (9)

with c [kPa] the total soil cohesion, σ [kPa] the total normal stress, and φ the total internal friction angle [deg].

The proximity of the anchorage zone to the top soil surface means there is not much normal stress from overburden. Also, a lot of agricultural soils have large silt- and clay-sized fractions, making their behavior, especially at higher degrees of saturation, more cohesive. Therefore, as a first approximation, the frictional component of the soil shear strength was assumed to be zero, which reduced the material response of the system to a single parameter, the total cohesion of the bulk soil. Finally, a complete mobilization of a uniform shear stress was assumed at all points of the interface, and the anchorage supply was described by this shear stress assuming the value of the total soil shear strength, i.e. the cohesion of the bulk soil. A balance of moments then expressed the anchorage strength [N*mm] in closed form as:

$$AS = \frac{π}{4} \cdot c \cdot {D}_{RB}^{3} \qquad ( 10 )$$

with the root ball diameter D_{RB} [mm] used to quantify the extent of the root-reinforced soil zone.

Use of this simplified anchorage framework allowed the soil strength for most soils prone to root lodging to be reasonably estimated via in situ measurement with an appropriately sized shear vane.

The anchorage supply sub-model was evaluated in closed form from the input parameters, namely the proximally measured bulk soil shear strength under appropriate moisture conditions, and the excavated root ball diameter, either measured directly or calculated from measurements of the root angle RA [deg] and structural rooting depth d_{SR} [cm] via:

D_{RB}=2·dₛᵣ·sin(RA/2) (11)

Once calculated, the ratio of the outputs of the AS and WD sub-models, respectively, quantified the model-predicted root lodging resistance per equation (1).

### Field Validation Experiments:

The accuracy of the root lodging model was assessed through field tests. Thirty mid-maturity maize hybrids with various phenotypic attributes and susceptibilities to root lodging were planted in randomized experimental blocks of 30 inch rows at a population density of 36,000 plants/acre at three research locations (Princeton Ill., Miami Mo., and Dallas Center Iowa). Plants were managed following standard practices. All locations experienced natural root lodging events at various times before flowering, while the plants were between the V7-V10 growth stages.

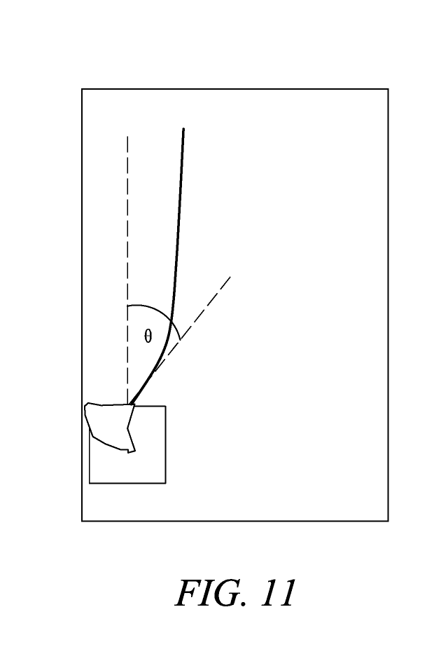

Plant phenotypes and location envirotypes were collected at each location following the lodging events. The severity of root lodging was measured by the field stalk angle [deg], defined as the angle from vertical of the base of the stalk (FIG. 11) within the plane of maximum lodging. This captured the amount of rotation by the root-soil support structure, quantifying the extent of anchorage failure. Plants were scored via a two-step process. First the entire row was quickly observed to coarsely quantify the total extent of lodging on a scale of 1-4; a score of 1 was assigned when most plants were completely vertical, and a score of 4 was assigned when most plants were significantly (>30 deg) lodged. Second, three plants were identified that were representative of the coarse row-level score. The field stalk angle was measured for these individuals with a digital angle-finder or inclinometer, and the plants were flagged for subsequent root excavation.

Soil envirotypes were measured at the same time as plant phenotypes, usually around a week after the lodging event. Consequently, the soil data described a different moisture state than when the lodging event occurred, and relative differences between plots under similar moisture conditions were emphasized. Soil measurements were made after the field stalk angles were measured and before root excavation, in the plane of lodging, 15 cm from the stalk base of flagged plants. The distance from the plants ensured that measurements characterized bulk (rather than root-reinforced) soil properties. Two measures of in situ soil strength were collected. First, the soil shear strength [kPa] was estimated using a Geovane shear vane with vane dimensions of 19 mm×38 mm, loaded at a rate of 0.8 (or, π/4) radians per second. The vane was inserted to a depth of 7 cm, to approximately coincide with the depth of the anchorage zone centroid. Second, the soil penetration resistance [MPa] was measured as a function of depth using an Eijkelkmap Penetrologger with a cone of 1 cm² base area and 60-degree angle inserted at a rate of 2 cm/s. Also, volumetric water content [%] of the top 6 cm was estimated via electrical permittivity measured with an ML3 Thetaprobe (Delta-T devices) connected to the penetrometer system.

Root phenotypes were measured from excavated plants. First, the top portion of each stalk was cut off just above the soil line to remove the visual indication of lodging severity, allowing subsequent root phenotypes to be taken under “blind” experimental conditions. Next, the root ball was excavated with a digging (“potato”) fork, inserted to fully cover the tines. This depth was sufficient to extract the full extent of root balls for all plants. The excavated root balls were soaked in a bucket of water for around thirty minutes, agitated to remove additional soil, and then characterized. Two root phenotypes were selected to describe the morphology of the root system, rather than individual roots. First, the root angle [deg] was estimated using a digital angle-finder. The timing of the lodging events meant that all excavated root systems were comprised of subterranean crown roots only; no above-ground brace (or, “prop”) roots had developed. This led to subjectivity in the angle measurements, as the generally ellipsoidal shape of the excavated root systems did not readily accommodate description by Euclidean geometry. The second root phenotype, the root ball diameter, was more appropriate for these morphologies. It was measured as the horizontally oriented diameter in the plane of lodging of the quasi-ellipsoidal root zone using a ruler. Initially, two orthogonally oriented measures of diameter were made, but this practice was abandoned when it was found that the additional data generally resided within measurement error.

Several above-ground phenotypes were measured. Plant height [cm] was measured as the base of the top (“flag”) leaf, using a ruler-stand. The stalk diameter [mm] at the base was measured using digital calipers as the average between the major and minor axes of the elliptical cross-section. Additional diameter measures were obtained just above and just below the ear, to define the stalk taper. Leaf area [cm²] was approximated as the area of the isosceles triangle formed by the leaf width and leaf length. Sampled leaves were selected at heights nearby where the ear height had been measured in previous seasons, to provide a data point close to the maximum area denoted in the distribution of FIG. 1a. Finally, several meteorological envirotypes were collected in the form of hourly measurements of precipitation [cm], average wind speed [m/s], and air temperature [° C.].

Results focused on the sensitivity and validation analyses. For both analyses, select phenotypes and envirotypes gathered from the field were assembled as inputs, while other input parameters were held constant at an assumed value due to lack of available data. Table 1 presents an exhaustive list of all input parameters, and categorizes them as either varying or fixed. Changes to the varying input parameters depended on the analysis.

TABLE 1

| Model input | | | | | | | |
|---|---|---|---|---|---|---|---|
| parameters | Typical | | | | Typical | | |
| Property | Value | Unit | Category | Property | Value | Unit | Category |
| Plant Height | 275 | cm | Varying | Avg wind speed | 15 | m/s | Varying |
| Ear Height | 105 | cm | Varying | Wind speed stdev | 1.5 | m/s | Fixed |
| Leaf Area | 430 | cm² | Varying | Turbulence length | 1.5 | m/s | Fixed |
| Leaf Drag | 0.15 | 1 | Varying | scale | | | |
| Total Leaf | 13 | 1 | Varying | Soil strength | 20 | kPa | Varying |
| Number | | | | Canopy diameter | 30 | cm | Fixed |
| Stalk Drag | 1 | 1 | Fixed | Root angle | 75 | deg | Varying |
| Ear Mass | 175 | gm | Varying | Root depth | 8 | cm | Varying |
| Stalk | 22 | mm | Varying | Air mass density | 1.25E− | gm/cc | Fixed |
| Diameter | | | | | 03 | | |
| Daily | 4 | cm | Varying | Internode flexural | 1800 | MPa | Fixed |
| rainfall | | | | modulus | | | |
| Damping | 0.1 | 1 | Fixed | Node flexural | 4500 | MPa | Fixed |
| ratio | | | | modulus | | | |

In the sensitivity analysis, all but one of the varying input parameters were held constant at their mean values while parameter of interest sequentially traversed the full range of its measured values. This allowed the model-predicted root lodging resistance to be calculated as a function of only the single varying input parameter. This was done for all varying input parameters, quantifying the sensitivity of the model to each one, and plotting model-predicted root lodging resistance versus the normalized range of the phenotype and envirotype intervals x^{(n)hd i}, calculated as:

$${x}^{{( n )}_{i}} = \frac{{x}_{i} - min ( x )}{max ( x ) - min ( x )} \qquad ( 12 )$$

with xᵢ the value of the phenotype or envirotype being normalized.

In the validation analysis, the varying input parameters took on the field-measured values for the hybrid being evaluated. All phenotypes and envirotypes were calculated as unweighted arithmetic means across the locations where they were collected. It is noted that results for some phenotypes and envirotypes at some locations were excluded from model validation due to data quality issues. Others that were difficult to measure and found not to be influential from the sensitivity study were kept constant at their average values from the sensitivity analysis, so as not to influence the ability of the model to describe the variability in root lodging response; treatment of input parameters is detailed in Table 1. Validation analysis showing field-measured lodging severity for each hybrid, averaged over three locations, plotted versus the biomechanical model-predicted root lodging resistance computed using average values of phenotypic and environmental input parameters from the three locations, showed good correlation (R-squared=0.5816) between the model predicted values and field measured values.

The sensitivity analysis showed that root lodging resistance is dominated by the anchorage components. This is seen from Table 2, which quantifies the influence of the phenotypes and envirotypes on root lodging resistance via the best fit linear slope obtained by plotting model-predicted root lodging resistance versus the normalized range of the phenotypes and envirotypes.

TABLE 2
Best fit linear slopes from sensitivity analysis

| Model Parameter | Influence |
|---|---|
| Root Angle | 99 |
| Root Depth | 94 |
| Soil Strength | 80 |
| Average Wind Speed | −73 |
| Plant Height | −23 |
| Leaf Drag Coefficient | −21 |
| Ear Height | −18 |
| Ear Mass | −9 |
| Stalk taper | 9 |
| Scaled Leaf Area | −8 |
| Stalk Base Diameter | 2 |
| Total Leaf Number | 1 |

The three anchorage components of root angle (99), root depth (94), and soil strength (80) were more influential than the primary wind demand component of wind speed (−73), while the most influential above-ground phenotypes of plant height (−23), leaf drag coefficient (−21), and ear height (−18) were clustered together as secondary effects. The relatively low values for leaf area (−8) and total leaf number (−1) suggest that the aerodynamic contributions of the leaves may have been suppressed by the drag reduction factor or insufficiently large values of the leaf drag coefficient, which was found to have more influence.

The validation analysis indicated that the biomechanical model described well the variation of natural root lodging measured in the field experiments. A negative linear relationship between the severity of lodging as quantified by the measured field stalk angle and model-computed lodging resistance was expected, and found to describe the data effectively. The residual of the linear trendline was evenly distributed over the range of comparison, showing little bias toward either highly resistant or susceptible genetics.

Validation of the present model suggested that the form of the anchorage sub-model (equation 10) is an effective tool for assessing lodging risk. The description of soil strength based on the sub-model may also be combined with other measured data for increased accuracy, including field elevation, which may be measured by LIDAR or on-board tractor GPS systems, slope stability analysis to vegetated hillsides, and the measured/derived hydrological properties of the soil, such as surface water flow, available water holding capacity and measured soil moisture. The method may be used to assess soil properties generally, since soil mechanical properties in combination with other measurements, such as soil hydrological properties and elevation, may provide important information about water movement and rate of flow that can be used to better predict water infiltration as versus water run-off.

### EXAMPLE 1

Soil measurements were taken to assess plant-relevant soil physical, mechanical, and soil hydrological properties in order to establish the validity of the approach of measuring soil mechanical properties via a proxy planter-mounted device that continuously collects data on soil mechanical resistance.

Before planting, data for soil physical, mechanical, and soil hydrological properties was manually collected in grids that were spatially dispersed throughout the field. Zone corner points were established with a high resolution GPS field unit, and internal points were established using survey equipment. Data was collected over a two-day period over which the soil moisture state did not appreciably change.

Dry soil bulk density was measured from cylindrical cores 2 inches in diameter and 3 inches in length, extracted from the soil surface. Cores were oven dried at 105 deg C. for 48 hours. The dried core material was used for texture analysis according standardized methods documented in ASTM D7928 and ASTM D6913. Organic matter content was measured via the loss on ignition method.

Soil shear strength was measured using a Geovane vane shear tester with vane dimensions 19×38 mm². The blade was inserted to a leading edge depth of 3 inches, and was turned at a rate of 0.8 rad/sec until failure occurred.

Saturated hydraulic conductivity (Ksat) was calculated using a Decagon dual-head infiltrometer with 5 cm insertion ring. Two pressure cycles were applied, with a high pressure head of 15 cm and low pressure head of 5 cm. Hold time at pressure for both was 20 minutes. Soak time was 15 minutes.

Penetration resistance was measured using an Eijkelkamp Penetrologger with #2 cone (2 cm2 base area). The unit logged the penetration force in depth increments of 1 cm. Rate of penetration was 2 cm/s. Total penetration depth was at least 30 cm. The penetration energy [J] was calculated as the area under the curve of penetration force vs. penetration depth up to 30 cm.

During planting, soil mechanical resistance was measured with a 20/20 SeedSense Gen2 aftermarket system from Precision Planting, Tremont, Ill. As described in US2010/0180695, the SeedSense unit includes a load sensing pin whose measurement is used by the seed planting assembly to determine the downforce present during planting. In this Example, this device was adapted to provide a measure of soil mechanical resistance as proof of concept of the methods described herein.

A wide degree of soil mechanical resistance variation was seen in the field, including from nearby areas of soil. This could be due to, for example, compaction, water flow patterns or past field use practices. Spatial averaging of the continuous soil mechanical resistance data revealed several relatively large and fairly uniform areas with different levels of soil mechanical resistance. This range of variation was unexpected large for this field, used for prior root lodging studies, because the field had been specifically managed for uniform soil characteristics to reduce variability. Nevertheless, areas of discretized regions, or sub-fields, of extremely hard soil (‘H’) with a high degree of soil mechanical resistance and extremely soft soil (‘S’) with a low degree of soil mechanical resistance were identified. This soil mechanical resistance data allowed identification of sub-fields that can be connected with soil compaction caused by regular year-over-year traffic of large equipment, which densified the soil to an extent that was not remediated by aggressive tillage treatments intended to increase structural uniformity. The soil mechanical resistance data could be utilized to optimize future field trafficking patterns to avoid this outcome. The soil mechanical resistance data also showed that the sub-fields can be subjected to different management approaches. For example, the geo-spatial coordinates of the hard sub-field could be used to define a region of the field that is subjected to enhanced tillage (depth, number of passes, etc) in order to remediate the harder soil structure. Or, for example, the soft sub-field could be subjected to a treatment with a land roller to densify the looser soil structure. The soil mechanical resistance data can also be used to alter planting density on the go. In accordance with the invention, a penetrometer or similar device may be positioned ahead of a planter, and the soil mechanical resistance data can be used as one or more components to make a real time planting density adjustment to the seed planter. For example, as the penetrometer passes over a zone identified as extremely hard, the data can be sent to a data file on the planter or in the cloud. If the region was identified as a zone with low drought potential, perhaps because of its GPS measured location, then the planter could automatically plant a larger density of plants in the identified soil compaction region, which could prevent root lodging. Alternatively, if the region was identified as a high drought potential, then planting a lower density of plants may be a better option.

The soundness of this approach was confirmed by the data. The soil mechanical resistance correlated well with soil bulk density, with water flow potential of the soil as measured by the saturated hydraulic conductivity (Ksat in centimeters per second), and with soil shear strength. Each approach showed clear variation of these values within the field, and as mentioned above, the variations seen in soil bulk density, saturated hydraulic conductivity and soil shear strength were present despite the fact that the field under study was being subjected to aggressive tillage procedures intended to homogenize the top level of soil structure for phenotypic screening of varietal differences in plant root lodging performance. The method documented herein, for extracting discrete measures of soil bulk density, saturated hydraulic conductivity and soil shear strength, from the continuous collection of soil mechanical resistance data will enable plant breeders to utilize this fast and convenient method to better account for varietal differences in root lodging performance. Differences in performance that had previously been attributed to genetics under the assumption of a uniform soil strength will be able to be more accurately partitioned to include variations of in-field soil strength.

In contrast, an analysis of the soil texture for sand, silt and clay showed that soil texture composition, as measured by the percentage of sand, clay and organic matter content, was surprisingly not a significant factor in predicting soil mechanical resistance.

While the methods and models described herein were optimized for maize, they may be adapted for use with the planting of any type of agricultural crop, including sorghum, wheat, rice, soybean, canola and cotton. Embodiments described herein are not intended to be limiting, and variations within the scope and spirit of the invention are encompassed herein.

What is claimed is:

1. A penetrometer for measuring soil characteristics in an agricultural field, said penetrometer comprising a rolling mechanism comprising a series of protruding surfaces operably connected to one or more load cells.

2. The penetrometer of claim 1, wherein the penetrometer comprises a load cell directly connected to a soil interacting part.

3. The penetrometer of claim 2, wherein the rolling mechanism is positioned on the row cleaning mount of a seed planting device.

4. The penetrometer of claim 1, wherein the rolling mechanism has a circumference equal to or less than 20 feet and comprises at least 4 load cells.

5. The penetrometer of claim 1, further comprising an accelerometer at or proximal to the center axis of the rolling mechanism.

6. The penetrometer of claim 1, further comprising an axial load cell on the mounting arm of the rolling mechanism, and wherein said axial load cell measures the soil mechanical resistance of the soil in response to the rolling mechanism.

7. The penetrometer of claim 1, wherein the series of protruding surfaces are conical.

8. A method of making real time planting density adjustment on a planter, comprising obtaining a reading from a penetrometer located on the planter in front of the planting assembly, comparing the penetrometer reading to a data file, and using the data file to direct real time planting density adjustments.

9. The method of claim 8, wherein the penetrometer comprises a load cell directly connected to a soil interacting part.

10. The method of claim 8, wherein the data file correlates the soil mechanical properties measured by the penetrometer to a root lodging risk assessment.

11. The method of claim 10, wherein the lodging risk is determined based on an anchorage supply sub-model that takes into account at least one of the predicted root angle, predicted root depth, or predicted root ball diameter.

12. The method of claim 11, wherein the predicted root angle, predicted root depth, or predicted root ball diameter is based on known characteristics of a seed variety.

13. A method of making real time planting density adjustments on a planter, comprising obtaining a continuous reading from a penetrometer or load sensing pin, comparing the reading to a data file, and using the data file to direct the planting density.

14. The method of claim 13, wherein the data file correlates the soil mechanical properties measured by the penetrometer or load sensing pin to a root lodging risk assessment.

15. The method of claim 14, wherein the lodging risk was determined based on an anchorage supply sub-model that takes into account at least one of the predicted root angle, predicted root depth, or predicted root ball diameter.

16. The method of claim 15, wherein the predicted root angle, predicted root depth, or predicted root ball diameter is based on known characteristics of a seed variety.

17. A method for the assessment of at least one soil property throughout an agricultural field, comprising collecting a continuous measurement of the mechanical resistance of the soil, wherein the soil property comprises at least one of soil bulk density or soil shear strength.

18. The method of claim 17, further comprising measuring at least one soil hydrological property.

19. The method of claim 17, wherein the continuous measurement of the mechanical resistance of the soil is obtained by a penetrometer comprising a load cell directly connected to a soil interacting part.

20. The method of claim 19, wherein the penetrometer further comprises a rolling mechanism comprising a series of protruding surfaces operably connected to one or more load cells on the rolling mechanism, and an axial load cell on the mounting arm of the rolling mechanism.

21. A method of making real time planting depth adjustment on a planter, comprising obtaining a reading from a soil moisture probe or a penetrometer having a direct connection between a load cell and a soil interacting part, comparing the soil moisture probe reading or penetrometer reading to a data file, and using the data file to direct real time planting depth adjustments.

22. The method of claim 21, wherein both the penetrometer reading and the soil moisture probe reading are utilized to direct real time planting depth adjustments.

23. The method of claim 21, further comprising utilizing one or both of an automated soil opening disc and an automated soil closing disc.

24. The method of claim 23, wherein the soil closing disc is automatically adjusted as the planter moves through the field to vary one or more of the depth or spacing of the closing discs based on the data file.

25. The method of claim 23, wherein the soil opening disc is automatically adjusted as the planter moves through the field to vary one or more of furrow depth or furrow width based on the data file.

26. The method of claim 25, wherein the soil closing disc is synchronized to close a furrow of equal depth and width to the furrow created by the opening disc.
